(19) United States
(12) Patent Application Publication
(10) Pub. No.: US 2025/0082271 A1
(43) Pub. Date: Mar. 13, 2025

(54) MEDICAL DEVICE SYSTEM

(71) Applicant: ViCentra B.V., Utrecht (NL)

(72) Inventor: Tim Oakes, Utrecht (NL)

(21) Appl. No.: 18/725,303

(22) PCT Filed: Dec. 14, 2022
(86) PCT No.: PCT/EP2022/085862

§ 371 Date: Jun. 28, 2024

(30) Foreign Application Priority Data
Jan. 6, 2022 (GB) 2200103.6

(51) Int. Cl. A61B 5/00; A61B 5/145; A61M 5/14; A61M 5/142

(52) U.S. Cl. CPC A61B 5/6833; A61B 5/14503; A61B 5/14532; A61M 5/1413; A61M 5/14248; A61B 2560/0443

(57) ABSTRACT

A medical device system, comprising: a disposable medical device part for sub-cutaneous drug infusion and/or sub-cutaneous analyte measurement, the disposable medical device part having a generally rigid housing and a first substrate, the first substrate having a first side with a first adhesive for securing to a patient's skin, and a second side attached to the generally rigid housing; and a second substrate having a first side with a second adhesive for contacting the patient's skin, and a second side for attaching to the first substrate, wherein the first adhesive is different to the second adhesive.

### FIELD OF THE INVENTION

The present invention relates to a medical device system, and a method of preparing the medical device system.

### BACKGROUND OF THE INVENTION

There has been a significant increase in the number of body-worn disposable medical devices adhesively attached to the skin for periods of time (typically days). The adhesives used can give rise to skin irritation and other skin issues, yet manufacturers are targeting longer wear times for these medical devices, and this will drive the use of more aggressive adhesives and likely cause an increase in skin issues. These disposable devices are typically manufactured at high volumes on automated manufacturing lines. The nature of this, in combination with strict medical device regulations, means that it is not typically viable for manufacturers to alter the disposable medical device, and this presents difficulties in providing users with a solution to any potential skin issues.

### SUMMARY OF THE INVENTION

A first aspect of the invention provides a medical device system, comprising: a disposable medical device part for sub-cutaneous or trans-cutaneous drug infusion and/or sub-cutaneous or trans-cutaneous analyte measurement, the disposable medical device part having a generally rigid housing and a first substrate, the first substrate having a first side with a first adhesive for securing to a patient's skin, and a second side attached to the generally rigid housing; and a second substrate having a first side with a second adhesive for contacting the patient's skin, and a second side for attaching to the first substrate, wherein the first adhesive is different to the second adhesive.

A further aspect of the invention provides a method of preparing the medical device system, comprising attaching the second side of the second substrate to the first side of the first substrate.

With this arrangement, a second adhesive can be used on the second substrate to adhesively attach to the patient's skin, with the second substrate acting as a barrier layer between the patient's skin and the first adhesive on the first substrate of the disposable medical device part. In this way, a different adhesive can be selected by the patient if the first adhesive on the disposable medical device part is likely to cause skin irritation or other skin issues.

The second substrate may comprise a through-hole and/or a slot for providing the disposable medical device part access to the patient's skin through the second substrate.

The disposable medical device part may comprise a sub-cutaneous element for sub-cutaneous drug infusion and/or sub-cutaneous analyte measurement extending through the through-hole or slot. The disposable medical device part may comprise a trans-cutaneous element for trans-cutaneous drug infusion and/or trans-cutaneous analyte measurement extending through the through-hole or slot.

The second substrate may comprise an alignment feature for indicating a position and/or an orientation of the disposable medical device part on the second substrate. Alternatively, or in addition, the second substrate may comprise an alignment feature for indicating a position and/or an orientation of an applicator for attaching the first substrate of the disposable medical device part to the second substrate.

The alignment feature may be a marking defining an outline of the disposable medical device part for guiding the positioning of the disposable medical device part on the second substrate. Alternatively, or in addition, the alignment feature may be a marking defining an outline of an applicator for attaching the first substrate of the disposable medical device part to the second substrate.

The second substrate may comprise alignment features such as a marking or holes that defines an outline of the disposable medical device part for indicating a position of the disposable medical device part and for guiding the positioning of the disposable medical device part on the second substrate.

The system may comprise an applicator for attaching the first substrate of the disposable medical device part to the second substrate, and wherein the second substrate comprises a marking or holes that define an outline of the applicator for indicating a position of the applicator and guiding the positioning of the applicator on the second substrate.

The outline of the disposable medical device part may be for indicating an orientation of the disposable medical device part and/or for indicating an orientation of an applicator

The second adhesive may be a silicone-based adhesive.

The first adhesive may be an acrylate adhesive.

The second adhesive may be an acrylate-based adhesive having a lower acrylate concentration than the first adhesive.

The second substrate may have a surface area greater than a surface area of the first substrate.

The surface area of the second substrate may be larger than the surface area of the first substrate, optionally at least 20% larger than the surface area of the first substrate, optionally at least 50% larger, and optionally at least 100% larger.

The first side of the second substrate may comprise a first portion comprising the second adhesive and a second portion absent of the second adhesive, and wherein the second portion extends up to an edge of the second substrate for providing an air channel between the second substrate and the patient's skin.

The second substrate may comprise a cut-out extending from an edge of the second substrate towards a centre of the second substrate so as to provide an air channel between the second substrate and the patient's skin.

The second substrate may comprise one or more breathability holes for providing an air gap between the first substrate and the patient's skin.

The medical device system may comprise one or more tabs extending from a main central portion of the second substrate.

The second substrate may comprise a first tab with an adhesive for attaching to a wire or a tube extending from the disposable medical device part and/or configured to attach to a second tab of the second substrate for encircling a wire or tube extending from the disposable medical device part.

The medical device system may comprise three or more tabs substantially evenly spaced about the second substrate.

At least a portion of the second substrate may comprise a reactive material arranged to indicate exposure to a fluid.

The fluid may be a medical drug.

The reactive material may be arranged to change colour and/or release an odour and/or change temperature when exposed to the fluid.

The second side of the second substrate may be attached to the first side of the first substrate via the first adhesive.

The medical device system may comprise two or more second substrates each comprising a different second adhesive to each of the other substrates, and preferably the medical device system comprises three or more substrates each comprising a different second adhesive to each of the other substrates.

The disposable medical device part may be an insulin pump or a glucose monitor.

### BRIEF DESCRIPTION OF THE DRAWINGS

Embodiments of the invention will now be described with reference to the accompanying drawings, in which:

FIG. 1a shows a disposable medical device system;

FIG. 1b shows a side view of the medical device system;

FIG. 1c shows a top view of first and second substrates of the medical device system;

FIG. 2 shows a slot formed in the first and second substrates for providing a sub-cutaneous element access to the patient's skin;

FIG. 3 shows an example in which the first substrate has substantially the same surface area as the surface area of the second substrate;

FIG. 4A shows a marking on the second substrate for guiding the positioning of the disposable medical device part onto the second substrate;

FIG. 4B shows a plurality of holes cut-out of the second substrate for guiding the positioning of the disposable medical device part on the second substrate;

FIG. 4C shows an arrow marking on the second substrate for assisting in the positioning of the disposable medical device part on the second substrate;

FIG. 4D shows an example in which the outer perimeter of the second substrate includes a notch for guiding the positioning and/or orientation of the disposable medical device part;

FIG. 4E shows an example in which the outer perimeter of the second substrate includes a tab for guiding the positioning and/or orientation of the disposable medical device part;

FIG. 5 shows an example in which the second substrate includes a plurality of tabs extending from a main central portion of the second substrate;

FIG. 6A shows an example in which the second substrate includes tabs arranged to wrap around at least a portion of the disposable medical device part;

FIG. 6B shows an example in which the tabs of the second substrate wrap around a tubing extending from the disposable medical device part;

FIG. 7 shows an example in which a portion of the second substrate includes a reactive material arranged to indicate exposure to a fluid;

FIG. 8A shows an example in which the second substrate includes a plurality of breathability holes for providing an air gap between the first substrate and the patient's skin;

FIG. 8b shows an example in which the second substrate includes breathability slots for providing an air gap between the first substrate and the patient's skin;

FIG. 9 shows an example in which a first portion of the second substrate is coated in the second adhesive, and a second portion of the second substrate is absent of the second adhesive;

FIG. 10 shows an example in which the disposable medical device part 2 is a sub-cutaneous infusion/injection device attachable to a pump, patient monitor, or other device;

FIG. 11A shows a second substrate for attaching two disposable medical device parts simultaneously;

FIG. 11B shows an example in which a sub-cutaneous element and associated device are attached separately to the same second substrate;

FIG. 12 shows an example in which the disposable medical device part is a glucose monitor;

FIG. 13 shows an example of an applicator for assisting the user in applying a disposable medical device part on the body;

FIG. 14 shows a second substrate shaped and sized to substantially match the shape and size of the applicator.

### DETAILED DESCRIPTION OF EMBODIMENT(S)

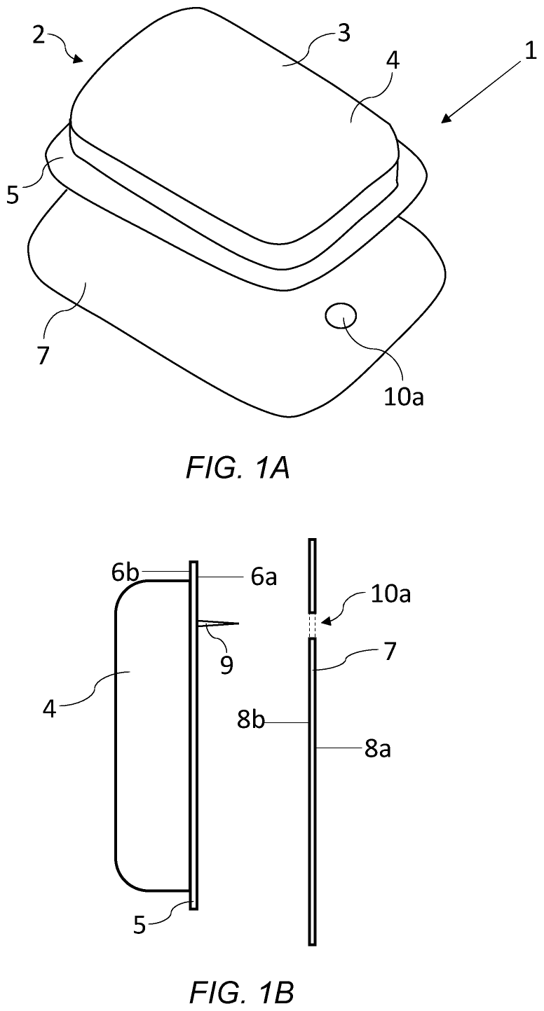

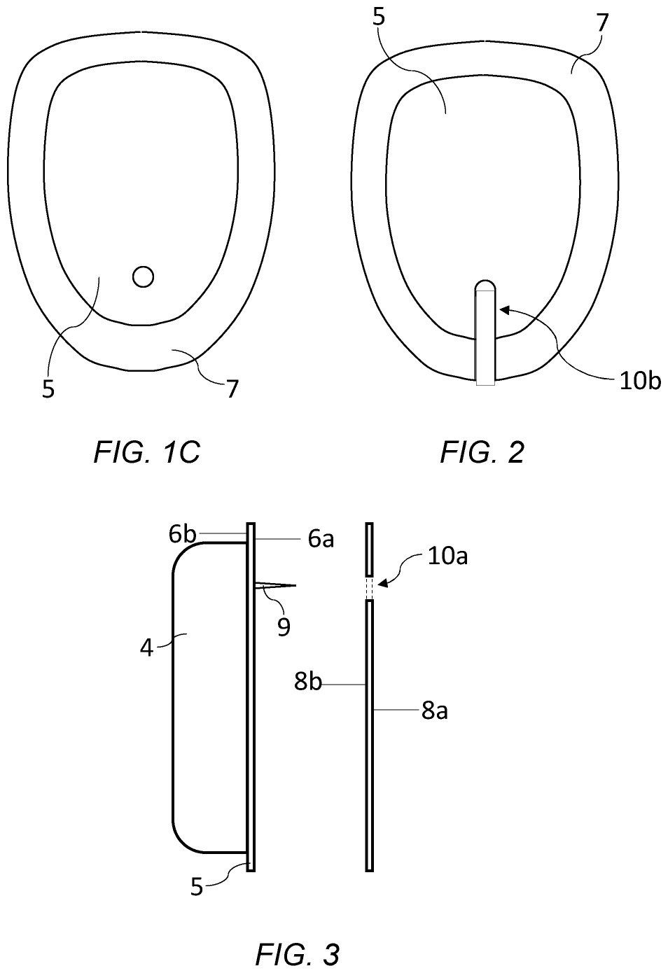

FIGS. 1A, 1B & 1C show an example of a medical device system 1. The medical device system 1 includes a disposable medical device part 2. The disposable medical device part 2 shown in FIG. 1A includes an insulin pump 3, however it will be understood that the invention is applicable to a wide range of disposable medical device parts 2 that can be adhesively attached to the skin of a patient. For example, a medical pump for delivering fluid and/or a drug to a patient, a patient monitoring device, such as a continuous glucose monitoring (CGM) device, or at least a part of such a system (e.g. a subcutaneous element connectable to a medical device).

The disposable medical device part 2 includes a generally rigid housing 4 and a first substrate 5. The first substrate 5 has a first side 6a with a first adhesive for securing to a patient's skin, and a second side 6b opposing the first side 6a that is attached to the generally rigid housing 4. A sub-cutaneous element 9 (such as a cannula) extends from the generally rigid housing 4 and through the first substrate 5, so as to be insertable into a patient. For example, the sub-cutaneous element 9 may be arranged for sub-cutaneous drug infusion or sub-cutaneous analyte measurement. Alternatively or in addition, the element 9 may be a trans-cutaneous element arranged for trans-cutaneous drug infusion or trans-cutaneous analyte measurement.

It is known in the art to attach the disposable medical device part 2 to a patient's skin via the first side 6a of the first substrate 5, and in particular via the first adhesive on the first side 6a of the first substrate 5. The first adhesive is typically an acrylate adhesive, or similar adhesive, that provides a long wear time for the disposable medical device part 2. However, these adhesives can cause skin irritation for some patient's, and this can discourage the use of such a disposable medical device part 2. Furthermore, manufacturers are targeting longer wear times, and this will encourage the use of more aggressive adhesives and likely cause an increase in skin issues.

The disposable medical device parts 2 are manufactured with the first substrate 5 pre-attached to the generally rigid housing 4, such that the patient has no option to swap the first adhesive with an alternative (e.g. less aggressive) adhesive.

According to an example of the invention, the medical device system 1 includes one or more second substrates 7 having a first side 8a with a second adhesive for contacting the patient's skin, and a second side 8b for attaching to the first substrate 5. In this way, the medical device system 1 according to the present invention may be considered as a kit of parts comprising a disposable medical device part 2 and one or more second substrates 7 attachable to the disposable medical device part 2. The patient may therefore select a second substrate 7 from the medical device system 1 that is suitable for their skin, as will be explained below.

The second adhesive on the second substrate 7 is different to the first adhesive on the first substrate 5. For example, the second adhesive may be a silicone-based adhesive, or an acrylate-based adhesive having a lower acrylate concentration than the first adhesive, or any other type of adhesive different to the first adhesive.

In examples in which the medical device system 1 includes two or more second substrates 7 (e.g. three, four or five substrates 7), each of the second substrates 7 may have a different second adhesive to each of the other second adhesives.

By providing a medical device system 1 with a second substrate 7 for positioning between the first substrate 5 and the patient's skin, the first adhesive on the first substrate 5 can be prevented from directly contacting the patient's skin. The patient may therefore select a second substrate 7 and attach the second side 8a of the second substrate 7 to the first side 6a of the first substrate 5.

As the medical device system 1 includes a (separate) second substrate 7, the patient is provided more flexibility in the choice of adhesive used to contact the patient's skin.

In some examples, the second substrate 7 may include a through-hole 10a or slot 10b (See the slot 10b in, e.g., FIG. 2) for providing the disposable medical device part 2, and in particular the sub-cutaneous element 9, access to the patient's skin through the second substrate 7. FIGS. 1A and 1B show the second substrate 7 including a through-hole 10a through which the sub-cutaneous element 9 is arranged to extend when the second substrate 7 is attached to the disposable medical device part 2. The through-hole 10a or slot 10b may assist in aligning the disposable medical device part 2 on the second substrate 7.

In the examples shown in FIGS. 1A-1C, the second substrate 7 has a surface area greater than the surface area of the first substrate 5. This is particularly illustrated by FIG. 1C, in which the generally rigid housing 4 is removed for clarity purposes, which shows the second substrate 7 extending beyond the edge of the first substrate 5. In the example shown in FIG. 1C, the surface area of the second substrate 7 is approximately 50% larger than the surface area of the first substrate 5, although the surface area of the second substrate 7 may be any suitable size greater than the first substrate 5, for example 10%, 30%, 70%, or 100% larger.

In examples providing a second substrate 7 with a larger surface area than the first substrate 5, a corresponding greater surface area is provided for the second adhesive to adhesively attach to the patient. As a result, adhesives with reduced adhesion may be used as the second adhesive. This may provide the patient with more viable options to use as the second adhesive, as the effects of the potentially reduced adhesion of the second adhesive can be mitigated.

However, it will be understood that the second substrate 7 may have the same, or a similar, surface area to that of the first substrate 5. FIG. 3 shows an example in which the first substrate 5 has substantially the same surface area as the surface area of the second substrate 7.

As previously discussed, the through hole 10a and/or slot 10b may function as alignment features for assisting in indicating a preferred position and/or orientation of the disposable medical device part 2 on the second substrate 7.

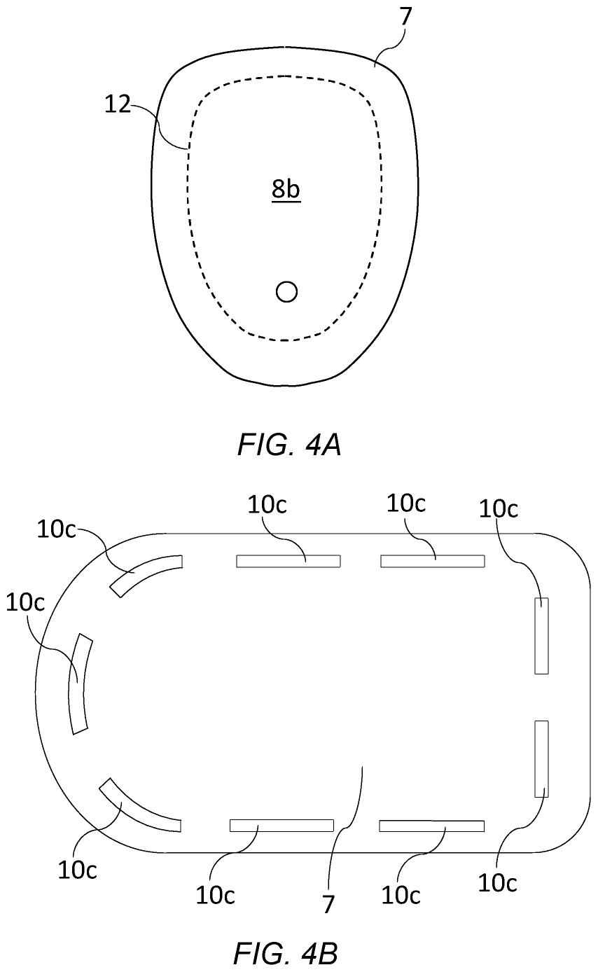

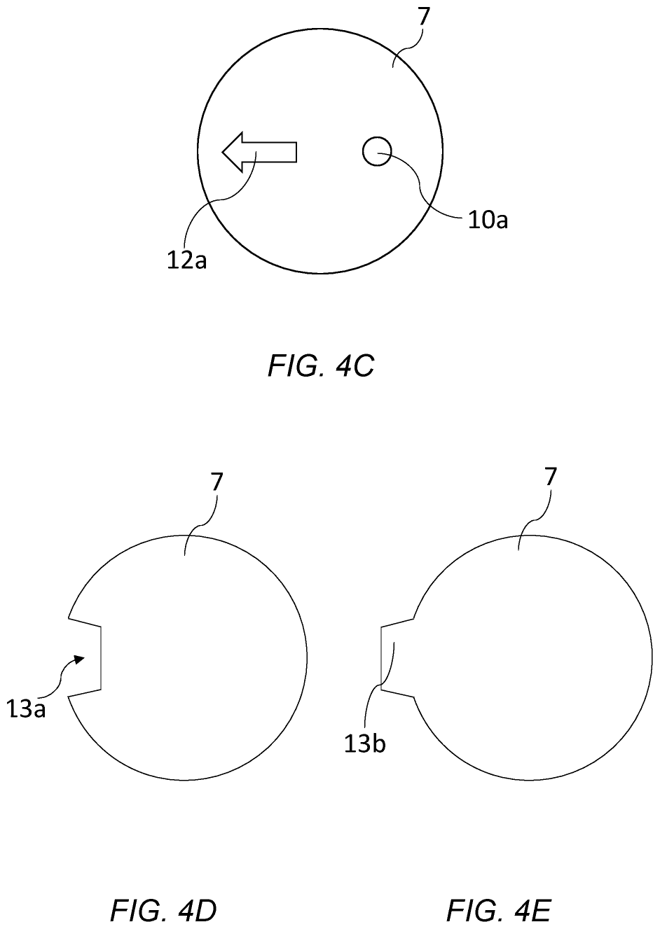

In addition or alternatively, the second substrate 7 may include other alignment features, such as shown in FIGS. 4A-4E.

In some examples, a marking 12 may define an outline of the disposable medical device part 2 for guiding the positioning of the disposable medical device part 2 onto the second substrate 7. FIG. 4A shows an example in which a marking 12 is visible on the second side 8b of the second substrate 7, wherein the marking 12 indicates an outline of an intended position of the disposable medical device part 2 on the second substrate 7.

In some examples, such as shown in FIG. 4B, the outline of the disposable medical device part 2 for guiding the positioning of the disposable medical device part 2 on the second substrate 7 may comprise one or more holes 10c cut-out of the second substrate 7

Alternatively, or in addition, the second substrate 7 marking 12 may indicate an orientation of the disposable medical device part 2. FIG. 4C shows an example comprising an arrow marking 12a. FIG. 4C also shows the through-hole 10a offset from a centre of the second substrate 7, and this asymmetry in at least some planes may additionally aid in the positioning of the disposable medical device part 2.

The outer perimeter of the second substrate 7 may additionally, or alternatively, guide the positioning of the disposable medical device part 2. FIG. 4D shows an example in which the second substrate 7 includes a notch 13a that may correspond to a similar notch (not shown) in the disposable medical device part 2, whilst FIG. 4E shows an example in which the second substrate 7 includes a tab 13b that may correspond to a similar tab (not shown) on the disposable medical device part 2.

It will be appreciated that the second substrate 7 may be any suitable shape to accommodate a particular disposable medical device part 2 or range of disposable medical device parts 2.

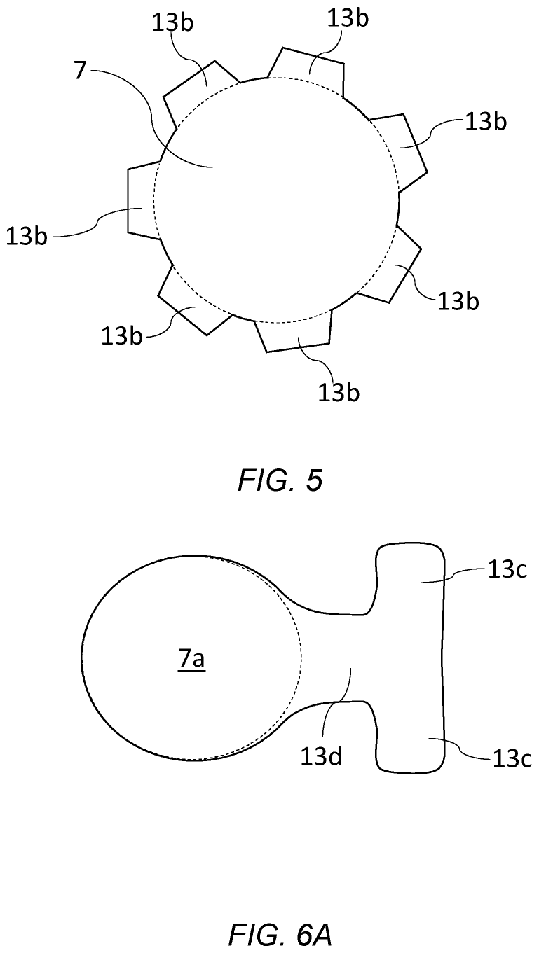

In some examples, the second substrate 7 may include two or more tabs 13b. FIG. 5 shows an example in which the second substrate includes seven tabs 13b substantially evenly spaced about the second substrate 7 and extending from a main central portion 7a of the second substrate 7. In this way, the tabs 13b may provide additional flexibility to the second substrate 7, assisting the second substrate 7 in conforming to the contours of the patient's skin and providing additional adhesive contact with the patient's skin.

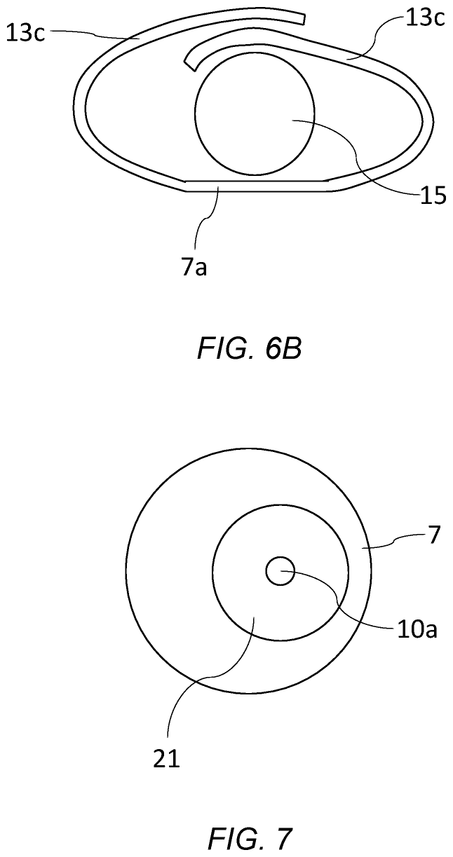

Alternatively, or in addition, the second substrate 7 may include tabs 13c for securing a wire or tubing 15 of the disposable medical device part 2 to the second substrate 7. FIGS. 6A & 6B show an example in which two tabs 13c extend from a tail portion 13d of the second substrate 7, wherein the two tabs 13c are configured to wrap around a tubing 15 extending from the disposable medical device part 2. In this way, additional control over the position and orientation of the wire or tubing 15 is provided.

One or both of the tabs 13c may fasten to the wire or tubing 15 (e.g. via adhesive). Alternatively, or in addition, the tabs 13c may fasten to each other. In some examples, at least a portion of a tab 13c may extend over the disposable medical device part 2 to assist in securing the position of the disposable medical device part 2 on the second substrate 7.

In some examples, such as shown in FIG. 7, at least a portion of the second substrate 7 may include a reactive material 21 arranged to indicate exposure to a fluid. For example, the reactive material 21 may change colour, release an odour when exposed to a fluid, or any other chemical reaction such as an exothermic or endothermic reaction, or a combination of the above. The fluid may be any fluid. Alternatively, the fluid may be a specific fluid, such as a medical drug.

In the example shown in FIG. 7, the reactive material 21 covers only a portion of the second substrate 7, and in particular a portion surrounding the through-hole 10a. In alternative examples, the reactive material 21 may cover substantially all of the second substrate 7, or around a perimeter of the second substrate 7.

In some examples, the second substrate 7 may include one or more holes 22a, slots 22b, portions of the second substrate 7 absent of the second adhesive, or combinations of the above, in order to improve breathability.

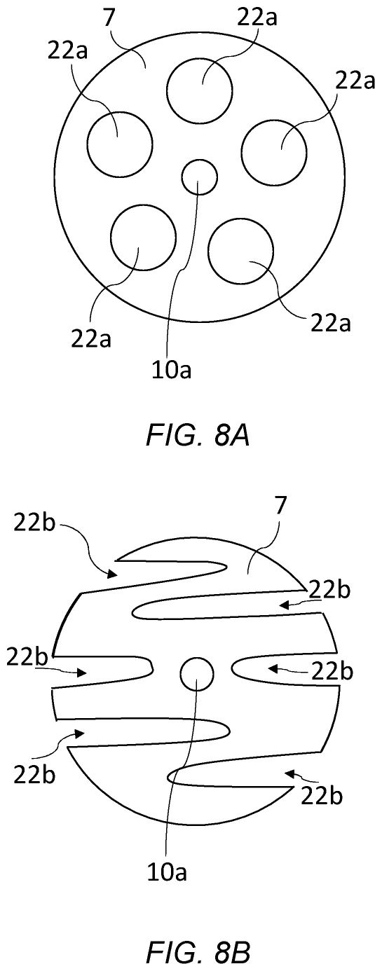

FIG. 8A shows an example in which the second substrate 7 includes a hole 10a for providing a sub-cutaneous element 9 access to the patient's skin through the second substrate 7. In addition, FIG. 8A shows five breathability holes 22a that provide an air gap between the first substrate 5 and the patient's skin. FIG. 8b shows an alternative example in which the second substrate 7 includes breathability slots 22b (alternatively referred to as cut-outs). The slots 22b extend from an edge of the second substrate 7, generally inwards towards a centre of the second substrate, so as to provide an air channel between the second substrate 7 and the patient's skin. It will be understood that the second substrate 7 may include any number of holes 22a and/or slots 22b, in any suitable configuration. The holes 22a and/or slots 22b may each be of different sizes and shaped, as required. The holes 22a and/or slots 22b may be suitable for simultaneously providing a sub-cutaneous element 9 access to the patient's skin through the second substrate 7 and providing an air gap between the first substrate 5 and the patient's skin.

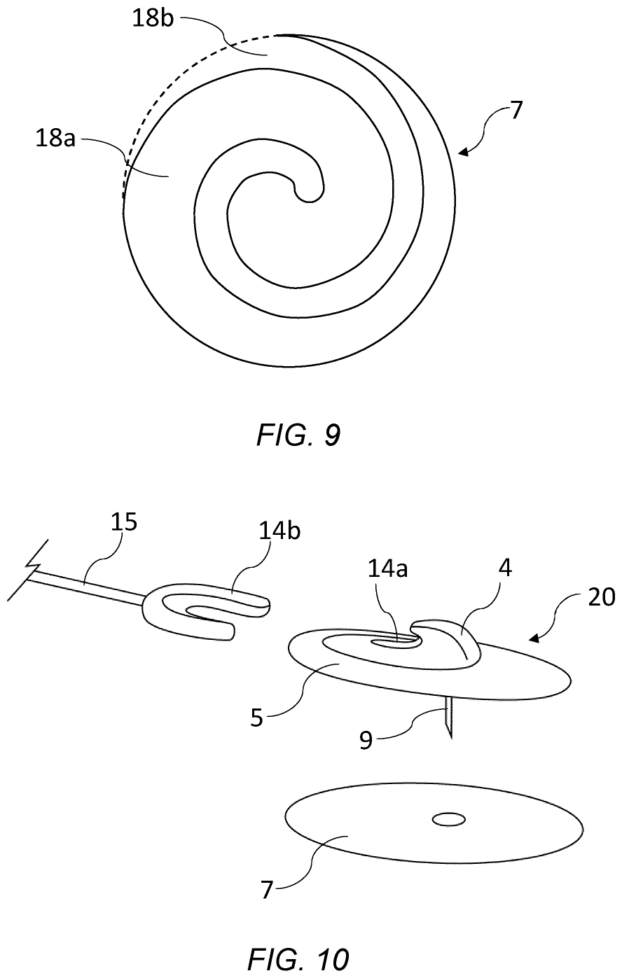

In some examples, additionally or as an alternative, breathability may be enhanced by omitting the second adhesive from portions of the second substrate 7. FIG. 9 shows an example in which a first portion 18a of the first side 8a of the second substrate 7 is coated in the second adhesive, and a second portion 18b of the first side 8a of the second substrate 7 is absent of the second adhesive. The second portion 18b may extend up to the edge of the second substrate 7 so as to provide an air channel between the second substrate 7 and the patient's skin that runs to an edge of the second substrate 7.

The example shown in FIG. 9 shows a continuous second portion 18b forming a path rotating about and towards a central axis of the second substrate 7, however it will be appreciated that the second portion 18b may take any suitable form, such as the holes 22a and slots 22b shown in FIGS. 8A & 8B.

In the example shown in FIG. 1A, the disposable medical device part 2 includes an insulin pump 3 with an integrated sub-cutaneous element 9. However, it will be appreciated that the disposable medical device part 2 may be any suitable pump for delivering fluid and/or a drug to a patient, or a patient monitoring device such as a continuous glucose monitoring (CGM) device. The disposable medical device part 2 may include an analyte sensor for measuring a parameter of a patient.

In some examples, the disposable medical device part 2 may be a sub-cutaneous and/or trans-cutaneous infusion/injection device 20 separate from any associated pump or patient monitoring device.

FIG. 10 shows such an example. In FIG. 10, the disposable medical device part 2 includes a connector 14a for connecting to a corresponding connector 14b and/or tubing 15 so as to convey a fluid (e.g. a drug or a patient's bodily fluid) to/from the disposable medical device part 2. For example, the tubing 15 may be connected to a pump, monitoring device or other device. The fluid may then be conveyed to the patient via the sub-cutaneous element 9.

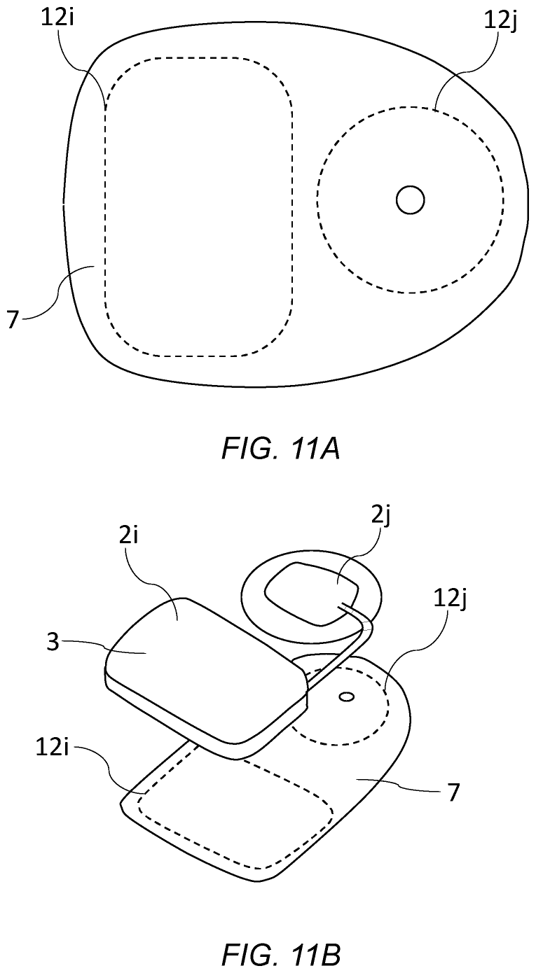

The device may be attached to a different, separate, second substrate 7. Alternatively, they may be positioned on the same second substrate 7, for example as shown in FIGS. 11A & 11B.

FIG. 11A shows a second substrate 7 arranged to accommodate two medical device parts 2i, 2j, in this example forming part of the same medical device system 1, in which one or both may be disposable. The second substrate 7 may include a first marking 12i arranged to accommodate a first medical device part 2i and a second marking 12j arranged to accommodate a second medical device part 2j. The markings 12i, 12j define an outline of the respective parts 2i, 2j, however it will be appreciated that any suitable marking 12 or indication of the position of the respective parts 2i, 2j may be provided.

FIG. 11B shows the first medical device part 2i, which may include an insulin pump 3, arranged to be positioned as indicated by the first marking 12i, and the second medical device part 2j, which may be a generally rigid housing 4 attached to a sub-cutaneous element 9, arranged to be positioned as indicated by the second marking 12j. In this example the second medical device part 2j is disposable and the first medical device part is reusable, although it will be appreciated that both may be disposable in some examples.

In some examples, the second substrate 7 may be attachable to two or more sub-cutaneous elements 9 simultaneously. In this case, the second substrate 7 may include a corresponding number of through-holes 10a and/or slots 10b.

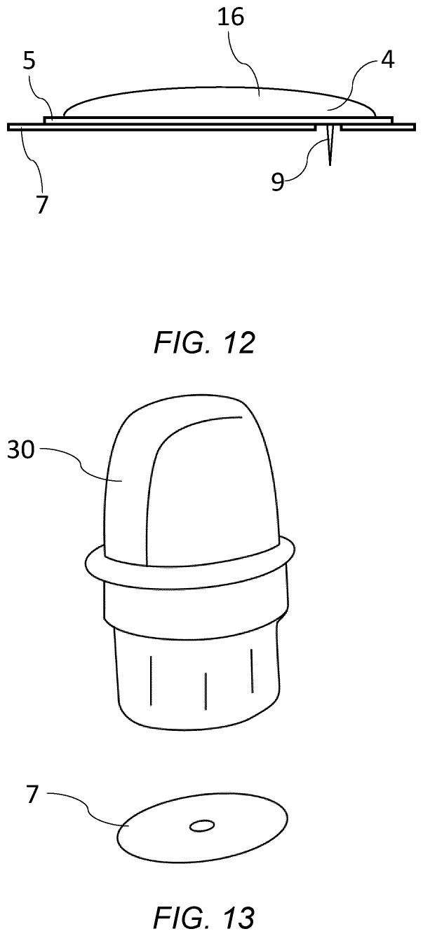

As previously discussed, the disposable medical device part 2 may be any suitable device. FIG. 12 shows an example in which the disposable medical device part 2 is a glucose monitor 16 arranged to monitor the glucose levels of a patient.

In the above examples, the second substrate 7 is attached to the disposable medical device part 2 prior to attaching the second substrate 7 to the skin of the patient. However, it will be appreciated that the second substrate 7 may be attached to the skin of the patient prior to attachment to the disposable medical device part 2. To assist in positioning the disposable medical device part 2 on the second substrate 7, the medical device system 1 may include an applicator 30, such as is known in the art. Although it will be appreciated that the disposable medical device part 2 may be applied without an applicator 30, e.g. by hand. An example of an applicator is shown in FIG. 13.

It will be clear to the skilled person that the examples described above may be adjusted or combined in various ways. In some examples, the second substrate 7 may include multiple alignment features (e.g. markings 12, 12a, 12i, 12j) corresponding to multiple disposable medical device parts 2 that can optionally be used with the same second substrate 7. The alignment features may overlap such that only a single of the disposable medical device parts 2 fits on the second substrate 7 at a given time. This may allow a second substrate 7 to be suitable for a range of disposable medical device parts 2.

In some examples, one or more of the alignment features described in the above examples may arranged to indicate a position and/or an orientation of the applicator 30 on the second substrate 7.

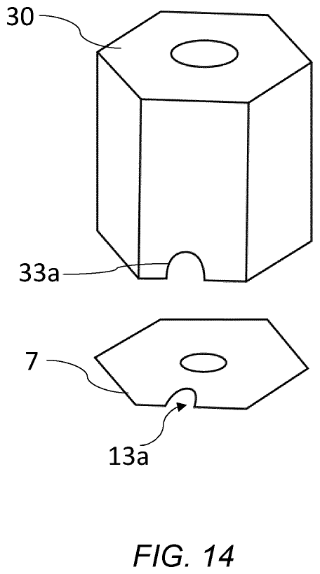

The outer edge of the second substrate 7 may be an alignment feature corresponding to the outer edge of the disposable medical device part 2 (and/or an associated applicator 30). In the example shown in FIG. 13, the applicator 30 is circular and the sub-cutaneous element 9 (not shown) is located centrally, with the second substrate 7 having a circular profile configured to substantially correspond to the circular shape of the applicator 30. In alternative examples, the second substrate 7 may be any suitable shape with any suitable alignment features to indicate a position and/or orientation of the disposable medical device part 2 or applicator 30. FIG. 14 shows an example in which the second substrate 7 is hexagon shaped, with a notch 13a to correspond with a corresponding notch 33a on the applicator 30.

It will be appreciated that the examples above are only exemplary, and that examples may combine one or more of the features described above. For example, any of the examples may comprise one or more of the through-holes 10a, 22a, slots 10b, 22b, markings 12, 12a, notches 13a, tabs 13b, 13c, reactive material 21, or portions absent of adhesive 18b that are described in the above examples. In addition, whilst the above-described examples refer to parts of a medical device system comprising a disposable medical device part for sub-cutaneous drug infusion and/or sub-cutaneous analyte measurement, it will be appreciated that the disposable medical device part may be equally suitable for trans-cutaneous drug infusion and/or trans-cutaneous analyte measurement. For instance, the element 9 may be a trans-cutaneous element.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. A medical device system, comprising:
a disposable medical device part for sub-cutaneous or trans-cutaneous drug infusion and/or sub-cutaneous or trans-cutaneous analyte measurement, the disposable medical device part having a generally rigid housing and a first substrate, the first substrate having a first side with a first adhesive for securing to a patient's skin, and a second side attached to the generally rigid housing; and a second substrate having a first side with a second adhesive for contacting the patient's skin, and a second side for attaching to the first substrate, wherein the first adhesive is different to the second adhesive.

2. The medical device system of claim 1,
wherein the second substrate comprises a through-hole and/or a slot for providing the disposable medical device part access to the patient's skin through the second substrate,
wherein the second substrate has a surface area greater than a surface area of the first substrate, and
(i) wherein the second substrate comprises a marking or holes that define an outline of the disposable medical device part for indicating a position of the disposable medical device part and guiding the positioning of the disposable medical device part on the second substrate; or
(ii) wherein the system comprises an applicator for attaching the first substrate of the disposable medical device part to the second substrate, and wherein the second substrate comprises a marking or holes that define an outline of the applicator for indicating a position of the applicator and guiding the positioning of the applicator on the second substrate

3. The medical device system of claim 1, wherein the second substrate comprises a through-hole and/or a slot for providing the disposable medical device part access to the patient's skin through the second substrate.

4. The medical device system of claim 2, wherein the disposable medical device part comprises a sub-cutaneous element for sub-cutaneous drug infusion and/or sub-cutaneous analyte measurement extending through the through-hole or slot and/or a trans-cutaneous element for trans-cutaneous drug infusion and/or trans-cutaneous analyte measurement extending through the through-hole or slot.

5. The medical device system of claim 1, wherein the second substrate comprises an alignment feature for indicating a position and/or an orientation of the disposable medical device part on the second substrate or an applicator for attaching the first substrate of the disposable medical device part to the second substrate.

6. The medical device system of claim 1, wherein the second substrate comprises a marking or holes defining an outline of the disposable medical device part for indicating a position of the disposable medical device part and for guiding the positioning of the disposable medical device part on the second substrate.

7. The medical device system of claim 1, comprising an applicator for attaching the first substrate of the disposable medical device part to the second substrate, and wherein the second substrate comprises a marking or holes that define an outline of the applicator for indicating a position of the applicator and guiding the positioning of the applicator on the second substrate.

8. The medical device system of claim 6, wherein the outline of the disposable medical device part is for indicating an orientation of the disposable medical device part and/or for indicating an orientation of an applicator.

9. The medical device system of claim 1, wherein the second adhesive is a silicone-based adhesive.

10. The medical device system of claim 1, wherein the first adhesive is an acrylate adhesive.

11. The medical device system of claim 10, wherein the second adhesive is an acrylate-based adhesive having a lower acrylate concentration than the first adhesive.

12. The medical device system of claim 1, wherein the second substrate has a surface area greater than a surface area of the first substrate.

13. (canceled)

14. The medical device system of claim 1, wherein the first side of the second substrate comprises a first portion comprising the second adhesive and a second portion absent of the second adhesive, and wherein the second portion extends up to an edge of the second substrate for providing an air channel between the second substrate and the patient's skin.

15. The medical device system of claim 1, wherein the second substrate comprises a cut-out extending from an edge of the second substrate towards a centre of the second substrate so as to provide an air channel between the second substrate and the patient's skin.

16. The medical device system of claim 1, wherein the second substrate comprises one or more breathability holes for providing an air gap between the first substrate and the patient's skin.

17. The medical device system of claim 1, comprising one or more tabs extending from a main central portion of the second substrate.

18. (canceled)

19. (canceled)

20. The medical device system of claim 1, wherein at least a portion of the second substrate comprises a reactive material arranged to indicate exposure to a fluid.

21. (canceled)

22. (canceled)

23. The medical device system of claim 1, wherein the second side of the second substrate is attached to the first side of the first substrate via the first adhesive.

24. The medical device system of claim 1, comprising two or more second substrates each comprising a different second adhesive to each of the other substrates, and preferably comprising three or more substrates each comprising a different second adhesive to each of the other substrates.

25. The medical device system of claim 1, wherein the disposable medical device part is an insulin pump or a glucose monitor.

26. (canceled)
